SEQUENCE LISTING (13 sequences)

SEQ ID NO: 1 - protein, 900 aa, Homo sapiens [sequence omitted]

SEQ ID NO: 2 - protein, 902 aa, Homo sapiens [sequence omitted]

SEQ ID NO: 3 - protein, 902 aa, Homo sapiens
MVLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVEKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNSGESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 915 aa, Homo sapiens
MVRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 917 aa, Homo sapiens [sequence omitted]

SEQ ID NO: 6 - protein, 917 aa, Homo sapiens
MVLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSGESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 902 aa, Homo sapiens
MVLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVEKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNSGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 915 aa, Homo sapiens; identical to SEQ ID NO: 4

SEQ ID NO: 9 - protein, 917 aa, Homo sapiens
MVRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVSGESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 917 aa, Homo sapiens [sequence omitted]

SEQ ID NO: 11 - protein, 915 aa, Homo sapiens
MVLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 917 aa, Homo sapiens; identical to SEQ ID NO: 6

SEQ ID NO: 13 - protein, 917 aa, Homo sapiens
MVLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2013/0243770 A1
(43) Pub. Date: Sep. 19, 2013

(54) TREATING DIABETES USING INHIBITORS OF IL-1

(71) Applicant: NOVO NORDISK A/S, Bagsvaerd (DK)

(72) Inventor: Jesper Lau, Farum (DK)

(73) Assignee: Novo Nordisk A/S, Bagsvaerd (DK)

(21) Appl. No.: 13/899,758

(22) Filed: May 22, 2013

Related U.S. Application Data
(63) Continuation of application No. 12/090,124, filed on Sep. 18, 2008, now abandoned.
(60) Provisional application No. 60/727,693, filed on Oct. 18, 2005.

(30) Foreign Application Priority Data
Oct. 14, 2005 (EP) 05109591.7

(51) Int. Cl. C07K 16/24

(52) U.S. Cl. CPC C07K 16/245
USPC 424/134.1; 424/136.1; 530/387.3

(57) ABSTRACT

The present invention describes a method of treating diabetes or metabolic syndrome with a compound that inhibits (a) IL-1, (b) the synthesis of IL-1, or (c) the release of IL-1.

### CROSS-REFERENCE TO RELATED APPLICATIONS

This application is a Continuation of U.S. application Ser. No. 12/090,124, filed Apr. 14, 2008, which is a 35 U.S.C. §371 national stage application of International Patent Application PCT/EP2006/067255 (published as WO 2007/042524), filed Oct. 11, 2006, which claimed priority of European Patent Application 05109591.7, filed Oct. 14, 2005; this application further claims priority under 35 U.S.C. §119 of U.S. of Provisional Application 60/727,693, filed Oct. 18, 2005.

### FIELD OF INVENTION

The present invention relates to treating type 1 or type 2 diabetes by administering a compound that inhibits (a) IL-1, (b) the synthesis of IL-1, (c) the release of IL-1. The present invention also relates to treating metabolic syndrome by administering a compound that inhibits (a) IL-1, (b) the synthesis of IL-1, (c) the release of IL-1.

### BACKGROUND OF THE INVENTION

Type 1 diabetes is characterised by a progressive loss of pancreatic beta cells due to an unfavourable balance between the destructive autoimmune processes targeting the beta cells on one side and the regenerative capacity of these cells on the other side. This imbalance eventually leads to total loss of beta cells and endogenous insulin secretion. Type 2 diabetes is characterised by insulin resistance and impaired beta cell function, which includes impaired first phase insulin release, reduced beta cell pulse mass and insulin deficiency (Donath & Halban, Diabetologia 2004, 47, 581-589.) This results in hyperglycemia that often is associated with the metabolic syndrome characterised by dyslipidemia, obesity, and hypertension. In the UKPDS study, it was found that beta cell function by the time of diagnosis of type 2 diabetes already is impaired, and that it continues to decline in spite of treatment. In time, loss of beta cell function will be accompanied and partly caused by a loss of beta cell mass, presumably due to apoptosis. (Butler, et. al. Diabetes 2003, 52, 102-110.) The decline in beta cell function and loss of beta cell mass could be caused by endoplasmic stress, by chronic hyperglycemia, (glucotoxicity), chronic hyperlipidemia (lipotoxicity), oxidative stress, beta amyloid fibrils, certain cytokines and adipokines, and a combination of these and other factors (Rhodes Science 2005, 307, 380-384).

It has been found that high concentrations of glucose or lipids, as well as human beta amyloid peptide, certain cytokines like IL-1β and certain adipokines like leptin, impair the function of rodent and human beta cells and cause apoptosis upon incubation in vitro. (See, Maedler, et. al. J. Clin. Invest. 2002, 110, 851-860; Maedler, et. al. Diabetes 2004, 53, 1706-1713; Ritzel, et. al. Diabetes 2003, 52, 1701-1708; Butler, et. al. Diabetes 2003, 52, 2304-2314 and Maedler, et al. Proc. Natl. Acad. Sci. U.S.A. 2004, 101, 8138-8143.) Incubation of human islets in the presence of high glucose concentrations or of leptin induces release of IL-1β to cause apoptosis of the beta cells. Neutralising the effect of IL-1β with a soluble IL-1 receptor antagonist (ILRa) has been shown to ameliorate the glucotoxicity and to protect from the deleterious effects of leptin. (See, Maedler, et. al. J. Clin. Invest. 2002, 110, 851-860; Maedler, et. al. Diabetes 2004, 53, 1706-1713; Proc. Natl. Acad. Sci. U.S.A. 2004, 101, 8138-8143; and, WO 04/002512.) IL-1β acts on IL-1β receptors on the beta cells and induces FAS expression, which subsequently cause apoptosis. Activation of the nuclear factor Kappa B (NFκB) is required for IL-1β induced FAS expression and apoptosis. NFκB transcription factors are composed of homo- and heterodimers of the Rel family of DNA-binding proteins. (Karin et al. Nat Rev Drug Discov 2004, 3, 17-26.) A key role of these transcription factors is to induce and coordinate the expression of a broad spectrum of pro-inflammatory genes including cytokines, chemokines, interferons, MHC proteins, growth factors, and cell adhesion molecules. NFκB is normally retained in the cytoplasm by IκB; however, upon cellular activation, IκB is phosphorylated by an IκB kinase (IKK) and is subsequently degraded. Free NFκB then translocates to the nucleus where it mediates pro-inflammatory gene expression. There are three classical IκB's: IκBα, IκBβ, and IκBε; all require the phosphorylation of two key serine residues before they can be degraded. Two major enzymes appear to be responsible for IκB phosphorylation: IKK-1 and IKK-2. Dominant-negative (DN) versions of either IKK-1 or IKK-2 (where ATP binding is disabled by the mutation of a key kinase domain residue) were found to suppress the activation of NFκB by TNF-a, IL-1b, LPS, and CD3/CD28 crosslinking; importantly IKK-2 DN was found to be a far more potent inhibitor than IKK-1 DN. Furthermore, the generation of IKK-1 and IKK-2 deficient mice has established the requirement of IKK-2 for activation of NFκB by pro-inflammatory stimuli and reinforced the dominant role of IKK-2 suggested by biochemical data. Indeed it was demonstrated that IKK-1 was dispensable for NFκB activation by these stimuli.

Anti-inflammatory salicylic acids have an antidiabetic effect in humans. (Yuan, et. al. Science 2001, 293, 1673-1677.) It furthermore has been found that signalling pathways leading to IKKβ and NFκB are activated in insulin responsive tissues of obese and high fat fed animals. It is therefore hypothesized that IKKβ/NFκB, which is part of the signalling pathway leading to the anti-inflammatory effects of salicylic acid, is part of the molecular mechanism leading to insulin resistance. (Yuan, et. al. Science 2001, 293, 1673-1677.) Activation of IKKβ/NFκB is in part mediated through activation of interleukin receptors by e.g., IL-1β and IL6. (Braddock, et. al. Nat Rev Drug Discov 2004, 3, 330-339.) IL-1β is a 17 kDa protein derived from the 31 kDa pro-IL-1β through cleavage by the IL-1β-converting enzyme (ICE or caspase-1). (Braddock, et. al. Nat Rev Drug Discov 2004, 3, 330-339.) Several signalling pathways regulate the transcriptional upregulation of pro-IL-1β including IL-1β itself via NFκB, TNFα, and Toll-like receptor ligands, such as lipopolysaccharide (LPS). Inhibitors of ICE will reduce LPS induced IL-1β release. It has been found that ICE is present in rat islets. (Karlsen, et. al. J. Clin. Endocrinol Metab 2000, 85, 830-836.)

Certain chemical entities, e.g., sulphonylureas, have furthermore been found to inhibit LPS induced IL-1β release, possibly through a mechanism that involves glutathione S-transferase (GST). (Braddock, et. al. Nat Rev Drug Discov 2004, 3, 330-339.) These are called Cytokine-Release Inhibitory Drugs (CRIDs).

In view of the above, it would appear beneficial to treat type 1 or type 2 diabetes or metabolic syndrome by either inhibiting IL-1β itself or inhibiting the synthesis or release of IL-1β. It would therefore also be desirable to treat type 1 or type 2 diabetes or metabolic syndrome in the manner described below in order to achieve one or more benefits such as improved potency, increased plasma halflife, lower therapeutic dose, fewer injections, lower production costs, and fewer side effects.

### SUMMARY OF THE INVENTION

In an aspect, the present invention provides a novel method of treating type 1 or type 2 diabetes by administering a compound that inhibits (a) IL-1, (b) the synthesis of IL-1, or (c) the release of IL-1.

In an aspect, the present invention provides a novel method of treating metabolic syndrome by administering a compound that inhibits (a) IL-1, (b) the synthesis of IL-1, (c) the release of IL-1.

In an aspect, the present invention provides a novel method of treating metabolic syndrome by administering a compound that inhibits (a) IL-1β, (b) the synthesis of IL-1β, (c) the release of IL-1β.

In an aspect, the present invention provides a novel method of treating metabolic syndrome by administering a compound that inhibits (a) IL-1α, (b) the synthesis of IL-1α, (c) the release of IL-1α.

In another aspect, the present invention provides a compound that inhibits (a) IL-1β, (b) the synthesis of IL-1β, or (c) the release of IL-1β for use in therapy.

In another aspect, the present invention provides a compound that inhibits (a) IL-1β, (b) the synthesis of IL-1β, or (c) the release of IL-1β for the manufacture of a medicament for the treatment of type 1 or type 2 diabetes.

In another aspect, the present invention provides a compound that inhibits (a) IL-1β, (b) the synthesis of IL-1β, or (c) the release of IL-1β for the manufacture of a medicament for the treatment of metabolic syndrome.

These and other objects, which will become apparent during the following detailed description, have been achieved by the inventors' discovery that compounds that inhibits (a) IL-1β, (b) the synthesis of IL-1β, or (c) the release of IL-1β or pharmaceutically acceptable salts thereof, should be effective for treating type 1 or type 2 diabetes or metabolic syndrome.

In another aspect, the present invention provides a compound that inhibits (a) IL-1α, (b) the synthesis of IL-1α, or (c) the release of IL-1α for use in therapy.

In another aspect, the present invention provides a compound that inhibits (a) IL-1α, (b) the synthesis of IL-1α, or (c) the release of IL-1α for the manufacture of a medicament for the treatment of type 1 or type 2 diabetes.

In another aspect, the present invention provides a compound that inhibits (a) IL-1α, (b) the synthesis of IL-1α, or (c) the release of IL-1α for the manufacture of a medicament for the treatment of metabolic syndrome.

These and other objects, which will become apparent during the following detailed description, have been achieved by the inventors' discovery that compounds that inhibits (a) IL-1α, (b) the synthesis of IL-1α, or (c) the release of IL-1α or pharmaceutically acceptable salts thereof, should be effective for treating type 1 or type 2 diabetes or metabolic syndrome.

### DESCRIPTION OF THE INVENTION

In an embodiment, the present invention provides a method of treating type 1 or type 2 diabetes, comprising: administering a compound that inhibits (a) IL-1, (b) the synthesis of IL-1 or (c) the release of IL-1.

Interleukin-1 (IL-1) traps are multimers of fusion proteins containing IL-1 receptor components and a multimerizing component capable of interacting with the multimerizing component present in another fusion protein to form a higher order structure, such as a dimer. Cytokine traps are a novel extension of the receptor-Fc fusion concept in that they include two distinct receptor components that bind a single cytokine, resulting in the generation of antagonists with dramatically increased affinity over that offered by single component reagents. In fact, the cytokine traps that are described herein are among the most potent cytokine blockers ever described. Briefly, the cytokine traps called IL-1 traps are comprised of the extracellular domain of human IL-1 R Type I (IL-1 RI) or Type II (IL-1RII) followed by the extracellular domain of human IL-1 Accessory protein (IL-1AcP), followed by a multimerizing component. In a preferred embodiment, the multimerizing component is an immunoglobulin-derived domain, such as, for example, the Fc region of human IgG, including part of the hinge region, the CH2 and CH3 domains. Alternatively, the IL-1 traps are comprised of the extracellular domain of human IL-1AcP, followed by the extracellular domain of human IL-1RI or IL-1 RII, followed by a multimerizing component. For a more detailed description of the IL-1 traps, see WO 00/18932, which publication is herein specifically incorporated by reference in its entirety. Preferred IL-1 traps have the amino acid sequence shown in SEQ ID NOs: 2, 4, 6, 8, 10, 12, 14, 16, 18, 20, 22, 24, and 26 of published US patent application US 2005/0129685 (corresponding to SEQ ID NO: 1-13 in the present application).

In specific embodiments, the IL-1 antagonist comprises an antibody fragment capable of binding IL-1α, IL-1β, IL-1 R1 and/or IL-1RAcp, or a fragment thereof. One embodiment of an IL-1 antagonist comprising one or more antibody fragments, for example, single chain Fv (scFv), is described in U.S. Pat. No. 6,472,179, which publication is herein specifically incorporated by reference in its entirety. In all of the IL-1 antagonist embodiments comprising one or more antibody-derived components specific for IL-1 or an IL-1 receptor, the components may be arranged in a variety of configurations, e.g., a IL-1 receptor component(s)-scFv(s)-multimerizing component; IL-1 receptor component(s)-multimerizing component-scFv(s); scFv(s)-IL-1 receptor component(s)-multimerizing component, etc., so long as the molecule or multimer is capable of inhibiting the biological activity of IL-1. In another embodiment, the IL-1 antagonist is IL-1ra.

In one preferred embodiment the IL-1 antagonist comprises an antibody domain capable of binding IL-1α, IL-1β, IL-1 R1 and/or IL-1RAcp. Domain Antibodies are the smallest functional binding units of antibodies, corresponding to the variable regions of either the heavy (VH) or light (VL) chains of human antibodies (Holt LJ in Trends in biotechnology, Vol. 21 (11), pp. 484-490, 2003). Domain Antibodies have a molecular weight of approximately 13 kDa. In contrast to conventional antibodies, Domain Antibodies are well expressed in bacterial, yeast, and mammalian cell systems. In addition, many Domain Antibodies are highly stable and retain activity even after being subjected to harsh conditions, such as freeze-drying or heat denaturation. These features make Domain Antibodies amenable to a wide range of pharmaceutical formulation conditions and manufacture processes. In addition, the small size of Domain Antibodies allows for higher molar quantities per gram of product, which should provide a significant increase in potency per dose and reduction in overall manufacturing cost. The Domain Antibodies selected against IL-1α, IL-1β, IL-1 R1 and/or IL-1RAcp can be used as a building block to create therapeutic products with unique characteristics not available to conventional antibodies or proteins, such as Dual Targeting Domain Antibodies that bind to two targets selected among IL-1α.alpha., IL-1β.beta., IL-1 R1 and/or IL-1RAcp in one easily produced molecule. Dual Targeting Dual Targeting Domain Antibodies that binds to IL-1a, IL-1β, IL-1 R1 or IL-1RAcp and to a plasma protein with a long circulating half live such as but not limited to albumin or transferrin and Domain Antibodies can be made with a tailored plasma half life. Long plasma half life can also be tailored by conjugation of a chemical moiety comprising a mono or poly disperse polyethyleneglycol group or an albumin binding moiety.

In a preferred embodiment an albumin or transferring binding Domain Antibody is conjugated or fused to IL-1Ra or a fragment or variant hereof.

In a preferred embodiment, the compound is an IL-1-specific fusion protein comprising two IL-1 receptor components and a multimerizing component, for example, an IL-1 trap described in U.S. patent publication No. 2003/0143697, published 31 Jul. 2003, herein specifically incorporated by reference in its entirety. In a specific embodiment, the IL-1 trap is the fusion protein shown in SEQ ID NO:2, 4, 6, 8, 10, 12, 14, 16, 18, 20, 22, 24, 26 of published US patent application US 2005/0129685 (corresponding to SEQ ID NO: 1-13 in the present application). A preferred IL-1 trap is shown in SEQ ID NO:10 of US 2005/0129685 (corresponding to SEQ ID NO: 5 in the present application). In specific embodiment, the compound is a modified IL-1 trap comprising one or more receptor components and one or more immunoglobulin-derived components specific for IL-1 and/or an IL-1 receptor. In another embodiment, the compound is a modified IL-1 trap comprising one or more immunoglobulin-derived components specific for IL-1 and/or an IL-1 receptor. In another embodiment, the IL-1 antagonist is a form of IL-1α, either being the full-length molecule or mature protein.

As used herein, “treating” or “treatment” covers the treatment of a disease-state in a mammal, particularly in a human, and includes: (a) preventing the disease-state from occurring in a mammal, in particular, when such mammal is predisposed to the disease-state but has not yet been diagnosed as having it; (b) inhibiting the disease-state, e.g., arresting or slowing its development; and/or (c) relieving the disease-state, e.g., causing regression of the disease state itself or some symptom(s) of the disease state.

As used herein, the terms “variant” or “variants” are intended to designate a protein wherein one or more amino acids of the parent protein have been substituted by another amino acid, and/or wherein one or more amino acids of the parent protein have been deleted, and/or wherein one or more amino acids have been inserted, and/or wherein one or more amino acids have been added to the parent protein. Such addition can take place either at the N-terminal end or at the C-terminal end of the parent protein or both.

One aspect of the present invention is thus a method of treating type 1 or type 2 diabetes, comprising: administering a compound that inhibits (a) IL-1, (b) the synthesis of IL-1, or (c) the release of IL-1, provided that the compound is an IL-1 Trap molecule.

In another aspect the invention provides a method of treating type 1 or type 2 diabetes, comprising: administering a compound that inhibits (a) IL-1β, (b) the synthesis of IL-1β, or (c) the release of IL-1β, provided that the compound is an IL-1 Trap molecule.

In another aspect the invention provides the use of an interleukin 1 neutralising molecule comprising one or two antibody fragments for the treatment of type 1 or type 2 diabetes. In another aspect the invention provides the use of an interleukin 1 neutralising molecule comprising a multifunctional antibody or antibody fragment for the treatment of type 1 or type 2 diabetes.

In another aspect the invention provides a molecule according to the aspect above where the antibody fragment(s) is a domain antibody.

In another aspect the invention provides a molecule according to the above aspects that comprises two different binding domains.

In another aspect the invention provides a molecule according to any one of the aspects above 6 that binds to IL-1R.

In another aspect the invention provides a molecule that binds to IL-1.

In another aspect the invention provides a molecule where the molecule is derivatised in such a way that plasma half-life is increased compared to the parent molecule.

In another aspect the invention provides a molecule where the molecule is chemically derivatised with a chemical moiety comprising a mono or poly disperse polyethylene-glycol group.

In another aspect the invention provides a molecule where the molecule is chemically derivatised or recombinantly fused with an albumin binding moiety.

In another aspect the invention provides a molecule where the albumin binding moiety is an antibody fragment.

In another aspect the invention provides a molecule where the albumin binding moiety is molecule with a molecular weight below 2000 dalton.

In another aspect the invention provides a molecule where the molecule is chemically derivatised or recombinantly fused with an IgG Fc domain.

In another aspect the invention provides the use of a molecule comprising an abumin binding Domain Antibody fused to IL-1Ra or a fragment or variant hereof for the treatment of type 1 or type 2 diabetes.

In another aspect the invention provides the use of a molecule comprising a transferring binding Domain Antibody fused to IL-1Ra or a fragment or variant hereof for the treatment of type 1 or type 2 diabetes.

In another aspect the invention provides a molecule comprising an abumin binding Domain Antibody fused to IL-1 binding Domain Antibody or variant hereof.

In another aspect the invention provides a molecule comprising a transferring binding Domain Antibody fused to IL-1 binding Domain Antibody or variant hereof.

### UTILITY

Inhibition of Release of IL-1β.

Assays for measuring release of IL-1β from different tissues, e.g., human blood, have been described. (Ichikawa, et. al. J. Antibiot (Tokyo) 2001, 54, 697-702 and Perregaux, et. al. J. Pharmacol Exp Ther 2001, 299, 187-197.)

Inhibition of Glucose Induced Beta Cell Death (Apoptosis).

Assays for measuring beta cell death/apoptosis induced by glucose have been described. (Maedler, et. al. J. Clin. Invest. 2002, 110, 851-860.)

Preservation of Beta Cell Function.

Assays for measuring effects of compounds on function of human beta cells incubated in presence of high glucose has been described. (See, Maedler, et. al. Diabetes 2004, 53, 1706-1713; Ritzel, et. al. J. Clin. Endocrinol Metab 2004, 89, 795-805; and, Bjorklund, et. al. Diabetes 2000, 49, 1840-1848.)

Acute Effects on Beta Cell Function In Vivo.

The acute effects of test compounds on beta cells function in vivo can be determined using an oral glucose tolerance test. The same method can be used to characterize the duration of action of the compounds.

Antidiabetic Effects.

The antidiabetic effects of the compounds can be determined can be measured using standard pharmacological methods. This includes measuring effects of the compounds on blood glucose and HbAlc upon acute and sub-chronic administration to diabetic rats and mice. Such methods have been described in the art.

Prevention of Diabetes.

Methods for measuring the ability of the compounds to prevent diabetes in preclinical models have been described in the art. This includes measuring blood glucose, HbAlc, and glucose tolerance in e.g., obese Zucker rats (Carr, et. al. Diabetes 2003, 52, 2513-2518) and in Psammomys Obeseus (Anis, et. al. Diabetologia 2004, 47, 1232-1244) to which the test compounds are administered.

Clinical Study. Treatment of Patients with Type 2 Diabetes Mellitus with Interleukin-1 Receptor Antagonist. (Suggested Study)

72 patients will be randomised according to a double-blind, placebo-controlled protocol in which half of the patients are treated with a compound of the invention, the other half with saline. The treatment period will last 13 weeks. This time-period should be sufficient for reversal of functional glucotoxicity (61) and feasible in terms of patient compliance. Whether 13 weeks of treatment will be sufficient to make significant changes in β-cell mass in unpredictable. However, blocking β-cell apoptosis, while new islet formation and β-cell replication are normal (62), may initiate enlargement of β-cell mass, which may progress beyond the treatment period. Patient evaluation will be performed at start and after 4, 13, 26, 39 and 52 weeks. Following 13 weeks, patients with a fasting plasma glucose levels>8 mM or with a glycosylated hemoglobin level (HbAlc)>8% will be treated with insulin. Insulin treatment will not be initiated earlier to avoid interference with possible effects of insulin on primary outcome in the period where the largest effect of the compound of the invention is expected. To assess effects of a compound of the invention on insulin sensitivity, a subset of 40 patients (20 receiving a compound of the invention and 20 placebo-treated) will undergo an euglycemic-hyperinsulinemic clamp as well as a muscle and fat biopsy at start and after the end of treatment (13 weeks).

### Inclusion Criteria:

### Age>30

Diabetes mellitus Type 2 (American Diabetes Association criteria) of at least 3 months duration and treated solely with diet and exercise and/or oral antidiabetic drugs.

### HbAlc>8%

Body-mass index (BMI)>27

### Exclusion Criteria

Positive GAD 2 or IA-2 antibodies
HbAlc>12%, polyuria and thirst (exclusion of severely decompensated patients)
Current treatment with insulin
Established anti-inflammatory therapy
CRP>30 mg/dl, fever, current treatment with antibiotics, or chronic granulomatous infections (e.g. tuberculosis) in the history or on a screening chest X-ray.
Neutropenia or anemia (leucocyte count<2.0×10⁹/l, haemoglobin<11 g/dl for males or <10 g/dl for females)
Pregnancy or breast-feeding
Severe liver or renal disease (AST or ALT>3 times the upper limit of normal laboratory range, serum creatinine>130 μM)
Ongoing malignant neoplasm
Use of any investigational drug within 30 days of enrolment into the study or within 5 half-lives of the investigational drug (whichever is the longer)

### Primary Endpoints:

Stimulated C-peptide and insulin (see below)

### HbAlc

Fasting plasma glucose (FPG)

### Secondary Endpoints:

Insulin requirement
Serum cytokines levels, CRP
Insulin secretion and Insulin-sensitivity index derived from an OGTT with insulin and glucose measurements.
In a subgroup of patients, insulin-sensitivity assessed by clamp techniques as well as by muscle and fat biopsies.

### Patient Evaluation

Patients will be evaluated as follows:
Physical examination including Body Mass Index, Waist to Hip Ratio, blood pressure (standing and supine), heart rate
Blood samples for determination of HbAlc, lipid profile including free fatty acids, HDL- and LDL-cholesterol, IL-1B, 1L-1Ra, IL-6, TNFα, CRP, sodium, potassium, creatinine, AST, ALT, and hematogramm.
24 h urine collection for albuminuria and creatinine clearance (only baseline and end of study).
Ophthalmologic examination including strereoscopic fundus photography (only baseline and end of study)
Standard oral glucose-tolerance-test (OGTT) with measurement of plasma blood glucose, insulin and C-peptide at 0, 30, 60, 90 and 120 min. At 120 min, 0.3 g/kg glucose+0.5 mg glucagon+5 g arginine will be injected intravenously followed by measurement of plasma blood glucose and insulin at 0, 3, 6, 9 and 12 min.
Weekly full blood glucose profile performed at home by the patient.
Euglycemic-hyperinsulinemic clamp and biopsies: a subset of 40 patients (20 receiving a compound of the invention and 20 placebo-treated) will undergo an euglycemic-hyperinsulinemic clamp as well as a muscle and fat biopsy. Polyethylene catheters will be placed in the antecubital vein for infusion and in the contralateral dorsal hand or antecubital vein for blood sampling. This “sampling” hand will be placed in a heated Plexiglas box to ensure arterialization of the venous blood sample. After an initial 40-min basal period, a primed-continuous insulin infusion (40 mU·m⁻²·min⁻¹) will be initiated and continued for 3 h. Basal and insulin stimulated steady state periods will be defined as the last 30 min of the 40 min basal state period and the last 30 min period of the 3 h clamp period. A variable infusion of glucose (180 g/l) will maintain euglycaemia during insulin infusion. Plasma glucose concentration will be monitored every 5 to 10 min during the basal and clamp periods using an automated glucose oxidation method. Blood samples will be drawn for measurements of insulin every 10 to 30 min during the basal and clamp steady state periods. Needle biopsies will be obtained in the basal state (time 0 min) from the vastus lateralis muscle and from the subcutaneous fat of the same region as well as from the abdominal region. The biopsies will be immediately frozen in liquid nitrogen and stored at −80° C. until analyzed for expression of cytokines (e.g. TNFα, IL-1α and β, IL-1Ra, IL-6, adiponectin and leptin) as well as for other genes and proteins of potential importance for insulin action.

The patients will be instructed to abstain from strenuous physical activity for 24 h and to fast for 9-10 h before both tests (OGTT and clamp studies). They should receive an injection of the study medication on study days but not other antidiabetic medications. The clamp will follow the OGTT, with a separation of 2 to 7 days. Study medication will be continued until the end of all assessments.

### Basic Medication:

Any change of patients' current therapy during the study should be avoided.
A compound of the invention could be given every 24 h in a single morning dose of 100 mg. The compound of the invention or placebo (saline) will be injected subcutaneously into the skin of the abdomen or upper thighs. The study nurse will instruct the patients how to perform the injections by themselves. One physician will always be available throughout 24 h for health or any other problems.

### Anticipated Conclusion

The following improvements can be expected in patients treated with a compound of the invention as compared to baseline or placebo-treated patients:
60% (or higher) increase in stimulated C-peptide and insulin levels.
Improvement of HbAlc: depending on baseline HbAlc, a decrease of HbAlc of 1% (baseline 8%) to 4% (baseline 12%).
Fasting plasma glucose (FPG): depending on baseline FPG, a decrease of FPG by 13% (baseline 8 mM glucose) to 27% (baseline 15 mM glucose).
No insulin requirement in the IL-1Ra-treated group versus 0.8 IU/Kg insulin in the placebo-treated.
60% (or higher) increase in insulin-sensitivity.
Normalisation of serum cytokines and CRP levels.

### Methods Demonstrating Efficacy:

### In Vitro Analysis of the Effect of the IL-1 Inhibiting Compounds on Beta-Cell Function and Viability:

Native islets or beta-cells of different species (e.g. Psammomys obesus and human) or beta-cell-lines (e.g. INS-1, RIN, MIN) are exposed to toxic concentrations of IL-1β or high glucose concentrations (e.g. 30 mmol) in the absence or presence of the IL-1 inhibiting compound. Following 1-6 days of culture the viability of the islets/cells are measured by standard commercially available viability assays (e.g. MTT, ATP. Caspase-3, LDH, PI, Tunnel assay) to demonstrate reduced toxic effect of high glucose or IL-1 in the absence of the IL-1 inhibiting compound. In addition the effect on beta-cell function is also addressed by measurement of the effect on insulin release.

### In Vivo Analysis of the Effect of the IL-1 Inhibiting Compounds on Treatment and Prevention of the Development of T2D:

Diabetes prone Psammomys obesus kept on a high energy diet until diabetes develops detected by blood glucose (approx 20 mmol) are divided into groups of vehicle treated animals and animals treated with the IL-1 inhibiting compounds (e.g. IL-1 Trap) at different concentrations for 2-4 additional weeks on the high energy diet. From the onset of treatment and onwards fasting morning blood glucose as well as HbAlC is measured to detect the ability of the IL-1 inhibitory strategy to normalize the T2D animals.

In a parallel experiment the animals are treated with vehicle or the IL-1 inhibiting compound along with the high energy diet so determine if the active compound (e.g. the IL-1 Trap) treatment can prevent the development of the T2D.

In another embodiment of the present invention, the present compounds are administered in combination with one or more further active substances in any suitable ratios. When used in combination with one or more further active substances, the combination of compounds is preferably a synergistic combination. Synergy occurs when the effect of the compounds when administered in combination is greater than the additive effect of the compounds when administered as a single agent. In general, a synergistic effect is most clearly demonstrated at sub-optimal concentrations of the compounds. Such further active agents may be selected from antidiabetic agents, antihyperlipidemic agents, anti-obesity agents, antihypertensive agents, and agents for the treatment of complications resulting from or associated with diabetes.

Suitable antidiabetic agents include insulin, GLP-1 (glucagon like peptide-1) derivatives such as those disclosed in WO 98/08871 (Novo Nordisk A/S), which is incorporated herein by reference, as well as orally active hypoglycemic agents.

Suitable orally active hypoglycemic agents preferably include imidazolines, sulfonylureas, biguanides, meglitinides, oxadiazolidinediones, thiazolidinediones, insulin sensitizers, α-glucosidase inhibitors, agents acting on the ATP-dependent potassium channel of the pancreatic β-cells e.g., potassium channel openers such as those disclosed in WO 97/26265, WO 99/03861 and WO 00/37474 (Novo Nordisk A/S) which are incorporated herein by reference, potassium channel openers, potassium channel blockers such as nateglinide or BTS-67582, glucagon antagonists such as those disclosed in WO 99/01423 and WO 00/39088 (Novo Nordisk A/S and Agouron Pharmaceuticals, Inc.), all of which are incorporated herein by reference, GLP-1 agonists such as those disclosed in WO 00/42026 (Novo Nordisk A/S and Agouron Pharmaceuticals, Inc.), which are incorporated herein by reference, DPP-IV (dipeptidyl peptidase-IV) inhibitors, PTPase (protein tyrosine phosphatase) inhibitors, inhibitors of hepatic enzymes involved in stimulation of gluconeogenesis and/or glycogenolysis, glucose uptake modulators, GSK-3 (glycogen synthase kinase-3) inhibitors, compounds modifying the lipid metabolism such as antihyperlipidemic agents and antilipidemic agents, compounds lowering food intake, and PPAR (peroxisome proliferator-activated receptor) and RXR (retinoid X receptor) agonists such as ALRT-268, LG-1268 or LG-1069.

In another embodiment of the present invention, the present compounds are administered in combination with a sulphonylurea, e.g., tolbutamide, chlorpropamide, tolazamide, glibenclamide, glipizide, glimepiride, glicazide, or glyburide.

In another embodiment of the present invention, the present compounds are administered in combination with a biguanide, e.g., metformin.

In another embodiment of the present invention, the present compounds are administered in combination with a meglitinide, e.g., repaglinide or senaglinide/nateglinide.

In another embodiment of the present invention, the present compounds are administered in combination with a thiazolidinedione insulin sensitizer, e.g., troglitazone, ciglitazone, pioglitazone, rosiglitazone, isaglitazone, darglitazone, englitazone, CS-011/CI-1037, T 174, the compounds disclosed in WO 97/41097 (DRF-2344), WO 97/41119, WO 97/41120, WO 00/41121. and WO 98/45292 (Dr. Reddy's Research Foundation), which are incorporated herein by reference.

In another embodiment of the present invention, the present compounds may be administered in combination with an insulin sensitizer, e.g., GI 262570, YM-440, MCC-555, JTT-501, AR-H039242, KRP-297, GW-409544, CRE-16336, AR-H049020, LY510929, MBX-102, CLX-0940, GW-501516, the compounds disclosed in WO 99/19313 (NN622/DRF-2725), WO 00/50414, WO 00/63191, WO 00/63192, WO 00/63193 (Dr. Reddy's Research Foundation), WO 00/23425, WO 00/23415, WO 00/23451, WO 00/23445, WO 00/23417, WO 00/23416, WO 00/63153, WO 00/63196, WO 00/63209, WO 00/63190, and WO 00/63189 (Novo Nordisk A/S), which are incorporated herein by reference.

In another embodiment of the present invention, the present compounds are administered in combination with an α-glucosidase inhibitor, e.g., voglibose, emiglitate, miglitol, or acarbose.

In another embodiment of the present invention, the present compounds are administered in combination with a glycogen phosphorylase inhibitor, e.g., the compounds described in WO 97/09040 (Novo Nordisk A/S).

In another embodiment of the present invention, the present compounds are administered in combination with an agent acting on the ATP-dependent potassium channel of the pancreatic β-cells, e.g., tolbutamide, glibenclamide, glipizide, glicazide, BTS-67582, or repaglinide.

In another embodiment of the present invention, the present compounds are administered in combination with nateglinide.

In another embodiment of the present invention, the present compounds are administered in combination with an antihyperlipidemic agent or a antilipidemic agent, e.g., cholestyramine, colestipol, clofibrate, gemfibrozil, lovastatin, pravastatin, simvastatin, probucol, or dextrothyroxine.

In another embodiment, the compounds of the present invention may be administered in combination with one or more anti-obesity agents or appetite regulating agents. Such agents may be selected from the group consisting of CART (cocaine amphetamine regulated transcript) agonists, NPY (neuropeptide Y) antagonists, MC3 (melanocortin 3) agonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 adrenergic agonists such as CL-316243, AJ-9677, GW-0604, LY362884, LY377267 or AZ-40140, MSH (melanocyte-stimulating hormone) agonists, MCH (melanocyte-concentrating hormone) antagonists, CCK (cholecystokinin) agonists, serotonin reuptake inhibitors (fluoxetine, seroxat or citalopram), serotonin and norepinephrine reuptake inhibitors, 5HT (serotonin) agonists, bombesin agonists, galanin antagonists, growth hormone, growth factors such as prolactin or placental lactogen, growth hormone releasing compounds, TRH (thyreotropin releasing hormone) agonists, UCP 2 or 3 (uncoupling protein 2 or 3) modulators, leptin agonists, DA (dopamine) agonists (bromocriptin, doprexin), lipase/amylase inhibitors, PPAR modulators, RXR modulators, TR β agonists, adrenergic CNS stimulating agents, AGRP (agouti related protein) inhibitors, H3 histamine antagonists such as those disclosed in WO 00/42023, WO 00/63208 and WO 00/64884, which are incorporated herein by reference, exendin-4, GLP-1 agonists, ciliary neurotrophic factor, and oxyntomodulin. Further anti-obesity agents are bupropion (antidepressant), topiramate (anticonvulsant), ecopipam (dopamine D1/D5 antagonist), and naltrexone (opioid antagonist).

In another embodiment of the present invention, the anti-obesity agent is leptin.

In another embodiment of the present invention, the anti-obesity agent is a serotonin and norepinephrine reuptake inhibitor, e.g., sibutramine.

In another embodiment of the present invention, the anti-obesity agent is a lipase inhibitor, e.g., orlistat.

In another embodiment of the present invention, the anti-obesity agent is an adrenergic CNS stimulating agent, e.g., dexamphetamine, amphetamine, phentermine, mazindol phendimetrazine, diethylpropion, fenfluramine, or dexfenfluramine.

In another embodiment of the present invention, the present compounds may be administered in combination with one or more antihypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. Further reference can be made to Remington: The Science and Practice of Pharmacy, 19th Edition, Gennaro, Ed., Mack Publishing Co., Easton, Pa., 1995.

In another embodiment of the present invention, the present compounds are administered in combination with insulin, insulin derivatives or insulin analogues.

In another embodiment of the present invention, the insulin is an insulin derivative is selected from the group consisting of B29-N^{ε}-myristoyl-des(B30) human insulin, B29-N^{ε}-palmitoyl-des(B30) human insulin, B29-N^{ε}-myristoyl human insulin, B29-N^{ε}-palmitoyl human insulin, B28-N^{ε}-myristoyl Lys^{B28} Pro^{B29} human insulin, B28-N^{ε}-palmitoyl Lys^{B28} Pro^{B29} human insulin, B30-N^{ε}-myristoyl-Thr^{B29}Lys^{B30} human insulin, B30-N^{ε}-palmitoyl-Thr^{B29}Lys^{B30} human insulin, B29-N^{ε}-(N-palmitoyl-γ-glutamyl)-des(B30) human insulin, B29-N^{ε}-(N-lithocholyl-γ-glutamyl)-des(B30) human insulin, B29-N^{ε}-(ω-carboxyheptadecanoyl)-des(B30) human insulin and B29-N^{ε}-(ω-carboxyheptadecanoyl) human insulin.

In another embodiment of the present invention, the insulin derivative is B29-N^{ε}-myristoyl-des(B30) human insulin.

In another embodiment of the present invention, the insulin is an acid-stabilised insulin. The acid-stabilised insulin may be selected from analogues of human insulin having one of the following amino acid residue substitutions:
A21G
A21G, B28K, B29P
A21G, B28D
A21G, B28E
A21G, B3K, B29E
A21G, desB27
A21G, B9E
A21G, B9D
A21G, B10E insulin.

In another embodiment of the present invention, the insulin is an insulin analogue. The insulin analogue may be selected from the group consisting of: an analogue wherein position B28 is Asp, Lys, Leu, Val, or Ala and position B29 is Lys or Pro; des(B28-B30); des(B27); or, des(B30) human insulin.

In another embodiment the analogue is an analogue of human insulin wherein position B28 is Asp or Lys, and position B29 is Lys or Pro.

In another embodiment the analogue is des(B30) human insulin.

In another embodiment the insulin analogue is an analogue of human insulin wherein position B28 is Asp.

In another embodiment the analogue is an analogue wherein position B3 is Lys and position B29 is Glu or Asp.

In another embodiment the GLP-1 derivative to be employed in combination with a compound of the present invention refers to GLP-1(1-37), exendin-4(1-39), insulinotropic fragments thereof, insulinotropic analogues thereof and insulinotropic derivatives thereof. Insulinotropic fragments of GLP-1(1-37) are insulinotropic peptides for which the entire sequence can be found in the sequence of GLP-1(1-37) and where at least one terminal amino acid has been deleted. Examples of insulinotropic fragments of GLP-1(1-37) are GLP-1(7-37) wherein the amino acid residues in positions 1-6 of GLP-1(1-37) have been deleted, and GLP-1(7-36) where the amino acid residues in position 1-6 and 37 of GLP-1(1-37) have been deleted. Examples of insulinotropic fragments of exendin-4(1-39) are exendin-4(1-38) and exendin-4(1-31). The insulinotropic property of a compound may be determined by in vivo or in vitro assays well known in the art. For instance, the compound may be administered to an animal and monitoring the insulin concentration over time. Insulinotropic analogues of GLP-1(1-37) and exendin-4(1-39) refer to the respective molecules wherein one or more of the amino acids residues have been exchanged with other amino acid residues and/or from which one or more amino acid residues have been deleted and/or from which one or more amino acid residues have been added with the proviso that said analogue either is insulinotropic or is a prodrug of an insulinotropic compound. Examples of insulinotropic analogues of GLP-1(1-37) are e.g. Met⁸-GLP-1(7-37) wherein the alanine in position 8 has been replaced by methionine and the amino acid residues in position 1 to 6 have been deleted, and Arg³⁴-GLP-1(7-37) wherein the valine in position 34 has been replaced with arginine and the amino acid residues in position 1 to 6 have been deleted. An example of an insulinotropic analogue of exendin-4(1-39) is Ser²Asp³-exendin-4(1-39) wherein the amino acid residues in position 2 and 3 have been replaced with serine and aspartic acid, respectively (this particular analogue also being known in the art as exendin-3). Insulinotropic derivatives of GLP-1 (1-37), exendin-4(1-39) and analogues thereof are what the person skilled in the art considers to be derivatives of these peptides, i.e., having at least one substituent which is not present in the parent peptide molecule with the proviso that said derivative either is insulinotropic or is a prodrug of an insulinotropic compound. Examples of substituents are amides, carbohydrates, alkyl groups and lipophilic substituents. Examples of insulinotropic derivatives of GLP-1(1-37), exendin-4(1-39) and analogues thereof are GLP-1(7-36)-amide, Arg³⁴, Lys²⁶(N^{ε}-(γ-Glu(N^{α}-hexadecanoyl)))-GLP-1(7-37) and Tyr³¹-exendin-4(1-31)-amide. Further examples of GLP-1(1-37), exendin-4(1-39), insulinotropic fragments thereof, insulinotropic analogues thereof and insulinotropic derivatives thereof are described in WO 98/08871, WO 99/43706, U.S. Pat. No. 5,424,286, and WO 00/09666.

In another embodiment of the present invention, the present compounds are administered in combination with more than one of the above-mentioned compounds, e.g. in combination with metformin and a sulphonylurea such as glyburide; a sulphonylurea and acarbose; nateglinide and metformin; acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

It should be understood that any suitable combination of the compounds according to the invention with diet and/or exercise, one or more of the above-mentioned compounds and optionally one or more other active substances are considered to be within the scope of the present invention. In another embodiment of the present invention, the pharmaceutical composition according to the present invention comprises e.g. a compound of the invention in combination with metformin and a sulphonylurea such as glyburide; a compound of the invention in combination with a sulphonylurea and acarbose; nateglinide and metformin; acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

### Pharmaceutical Compositions

Pharmaceutical compositions containing a compound according to the present invention may be prepared by conventional techniques, e.g. as described in Remington's Pharmaceutical Sciences, 1985 or in Remington: The Science and Practice of Pharmacy, 19th edition, 1995.

One object of the present invention is to provide a pharmaceutical formulation comprising a compound according to the present invention which is present in a concentration from about 0.1 mg/ml to about 25 mg/ml, and wherein said formulation has a pH from 2.0 to 10.0. The formulation may further comprise a buffer system, preservative(s), isotonicity agent(s), chelating agent(s), stabilizers and surfactants. In one embodiment of the invention the pharmaceutical formulation is an aqueous formulation, i.e. formulation comprising water. Such formulation is typically a solution or a suspension. In a further embodiment of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment the pharmaceutical formulation is a freeze-dried formulation, whereto the physician or the patient adds solvents and/or diluents prior to use.

In another embodiment the pharmaceutical formulation is a dried formulation (e.g. freeze-dried or spray-dried) ready for use without any prior dissolution.

In a further aspect the invention relates to a pharmaceutical formulation comprising an aqueous solution of a compound according to the present invention, and a buffer, wherein said compound is present in a concentration from 0.1 mg/ml or above, and wherein said formulation has a pH from about 2.0 to about 10.0.

In another embodiment of the invention the pH of the formulation is from about 7.0 to about 9.5. In another embodiment of the invention the pH of the formulation is from about 3.0 to about 7.0. In another embodiment of the invention the pH of the formulation is from about 5.0 to about 7.5. In another embodiment of the invention the pH of the formulation is from about 7.5 to about 9.0. In another embodiment of the invention the pH of the formulation is from about 7.5 to about 8.5. In another embodiment of the invention the pH of the formulation is from about 6.0 to about 7.5. In another embodiment of the invention the pH of the formulation is from about 6.0 to about 7.0.

In another embodiment of the invention the pH of the formulation is from about 3.0 to about 9.0, and said pH is at least 2.0 pH units from the isoelectric pH of compound of the present invention.

In a further embodiment of the invention the buffer is selected from the group consisting of sodium acetate, sodium carbonate, citrate, glycylglycine, histidine, glycine, lysine, arginin, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment of the invention the formulation further comprises a pharmaceutically acceptable preservative. In a further embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 19th edition, 1995.

In a further embodiment of the invention the formulation further comprises an isotonic agent. In a further embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol)polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galacititol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 19th edition, 1995.

In a further embodiment of the invention the formulation further comprises a chelating agent. In a further embodiment of the invention the chelating agent is selected from salts of ethylenediaminetetraacetic acid (EDTA), citric acid, and aspartic acid, and mixtures thereof. In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 19th edition, 1995.

In a further embodiment of the invention the formulation further comprises a stabiliser. The use of a stabilizer in pharmaceutical compositions is well-known to the skilled person. For convenience reference is made to Remington: The Science and Practice of Pharmacy, 19th edition, 1995.

More particularly, compositions of the invention are stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L, D, or DL isomer) of a particular amino acid (e.g. glycine, methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include S-ethyl homocysteine and S-butyl homocysteine and suitable cystein analogues include S-methyl-L cystein. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention methionine (or other sulphur containing amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L, D, or DL isomer) or combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In a further embodiment of the invention the formulation further comprises a stabiliser selected from the group of high molecular weight polymers or low molecular compounds. In a further embodiment of the invention the stabilizer is selected from polyethylene glycol (e.g. PEG 3350), polyvinylalcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing.

In a further embodiment of the invention the formulation further comprises a surfactant. In a further embodiment of the invention the surfactant is selected from a detergent, ethoxylated castor oil, polyglycolyzed glycerides, acetylated monoglycerides, sorbitan fatty acid esters, polyoxypropylene-polyoxyethylene block polymers (e.g. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lecitins and phospholipids (e.g. phosphatidyl serine, phosphatidyl choline, phosphatidyl ethanolamine, phosphatidyl inositol, diphosphatidyl glycerol and sphingomyelin), derivates of phospholipids (e.g. dipalmitoyl phosphatidic acid) and lysophospholipids (e.g. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (e.g. cephalins), glyceroglycolipids (e.g. galactopyransoide), sphingoglycolipids (e.g. ceramides, gangliosides), dodecylphosphocholine, hen egg lysolecithin, fusidic acid derivatives- (e.g. sodium tauro-dihydrofusidate etc.), long-chain fatty acids and salts thereof C6-C12 (e.g. oleic acid and caprylic acid), acylcarnitines and derivatives, Nα-acylated derivatives of lysine, arginine or histidine, or side-chain acylated derivatives of lysine or arginine, Nα-acylated derivatives of dipeptides comprising any combination of lysine, arginine or histidine and a neutral or acidic amino acid, Nα-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulfate or sodium lauryl sulfate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quarternary ammonium bases) (e.g. cetyl-trimethylammonium bromide, cetylpyridinium chloride), non-ionic surfactants (e.g. dodecyl β-D-glucopyranoside), poloxamines (e.g. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 19th edition, 1995.

It is possible that other ingredients may be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatin or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing a compound according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the compound, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the formulation of solids, semisolids, powder and solutions for pulmonary administration of the compound, using, for example a metered dose inhaler, dry powder inhaler and a nebulizer, all being devices well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-cystallization, precipitation, co-precipitation, emulsification, dispersion, high pressure homogenization, encapsulation, spray drying, microencapsulation, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000). Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the compound according to the present invention in the form of a nasal or pulmonal spray. As a still further option, the pharmaceutical compositions containing the compound of the invention can also be adapted to transdermal administration, e.g. by needle-free injection or from a patch, optionally an iontophoretic patch, or transmucosal, e.g. buccal, administration.

The term “stabilized formulation” refers to a formulation with increased physical stability, increased chemical stability or increased physical and chemical stability.

The term “physical stability” of the protein formulation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to protein aggregation, when it shows visual turbidity in daylight. Alternatively, the turbidity of the formulation can be evaluated by simple turbidity measurements well-known to the skilled person. Physical stability of the aqueous protein formulations can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as anthracene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein formulation as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized formulation” refers to a formulation with increased physical stability, increased chemical stability or increased physical and chemical stability. In general, a formulation must be stable during use and storage (in compliance with recommended use and storage conditions) until the expiration date is reached.

In one embodiment of the invention the pharmaceutical formulation comprising the compound according to the present invention is stable for more than 6 weeks of usage and for more than 3 years of storage.

In another embodiment of the invention the pharmaceutical formulation comprising the compound according to the present invention is stable for more than 4 weeks of usage and for more than 3 years of storage.

In a further embodiment of the invention the pharmaceutical formulation comprising the compound according to the present invention is stable for more than 4 weeks of usage and for more than two years of storage.

In an even further embodiment of the invention the pharmaceutical formulation comprising the compound is stable for more than 2 weeks of usage and for more than two years of storage.

While the invention has been described and illustrated with reference to certain preferred embodiments thereof, those skilled in the art will appreciate that various changes, modifications, and substitutions can be made therein without departing from the spirit and scope of the present invention. For example, effective dosages other than the preferred dosages as set forth herein may be applicable as a consequence of variations in the responsiveness of the mammal being treated for type 2 diabetes. Likewise, the specific pharmacological responses observed may vary according to and depending on the particular active compound selected or whether there are present pharmaceutical carriers, as well as the type of formulation and mode of administration employed, and such expected variations or differences in the results are contemplated in accordance with the objects and practices of the present invention.

What is claimed is:

1. A method of treating diabetes in a mammal comprising administering an amount of an IL-1 Trap molecule to the mammal that is effective to inhibit (a) IL-1, (b) the synthesis of IL-1, or (c) the release of IL-1.

2. The method of claim 1, wherein the IL-1 trap inhibits (a) IL-1β, (b) the synthesis of IL-1β, or (c) the release of IL-1β.

3. A method of treating diabetes comprising administering to a mammal an effective amount of (a) a multifunctional antibody that binds IL-1R and IL-1 or (b) a combination of antibodies that bind IL-1R and IL-1.

4. The method of claim 3, wherein the method comprises administering a multifunctional antibody that binds IL-1R and IL-1.

5. The method of claim 3, wherein the method comprises administering a combination of antibodies that bind IL-1R and IL-1.

6. The method of claim 1, wherein the IL-1 Trap is chemically derivatized with a chemical moiety comprising a mono- or poly-disperse polyethyleneglycol group.

7. The method of claim 1, wherein the IL-1 Trap is chemically derivatized or recombinantly fused with an albumin binding moiety.

8. The molecule according to claim 11, wherein the albumin binding moiety is an antibody fragment.

9. The method of claim 1, wherein the IL-1 Trap is chemically derivatized or recombinantly fused with an IgG Fc domain.

10. A molecule comprising an albumin binding Domain Antibody fused to an IL-1 binding Domain Antibody.

11. A molecule comprising a transferin binding Domain Antibody fused to an IL-1 binding Domain Antibody.

12. The method of claim 8, wherein the treatment consists of inhibiting or relieving diabetes in the mammal.

13. The method of claim 9, wherein the treatment consists of inhibiting or relieving diabetes in the mammal.

14. The method of claim 1, wherein the treatment consists of inhibiting or relieving diabetes in the mammal.
